(19) United States
(12) Patent Application Publication
(10) Pub. No.: US 2024/0203554 A1
(43) Pub. Date: Jun. 20, 2024

(54) MULTI-PLATFORM PRESCRIPTION DRUG CLAIM ADJUDICATION

(71) Applicant: Express Scripts Strategic Development, Inc., St. Louis, MO (US)

(72) Inventors: Arun Siva, Shrewsbury, MA (US); Yevgeniya Kuznetsova, Montville, NJ (US); Damayanti Majumdar, Eden Prairie, MN (US); KanmaniGowri Jeyaram, Belle Mead, NJ (US); Ankur Kaneria, Cedar Park, TX (US); Harry S. Gangaikondan-Iyer, Morris Plains, NJ (US)

(21) Appl. No.: 18/537,901

(22) Filed: Dec. 13, 2023

Related U.S. Application Data
(60) Provisional application No. 63/433,251, filed on Dec. 16, 2022.

(51) Int. Cl. G16H 20/10; G06N 3/08; G16H 10/60

(52) U.S. Cl. CPC G16H 20/10; G06N 3/08; G16H 10/60

(57) ABSTRACT

Methods and systems for performing multi-platform prescription drug claim adjudication are provided. The methods and systems perform operations comprising: receiving, by a first server associated with a first claim adjudication entity, a claim for a prescription drug for a patient, the first claim adjudication entity managing a prescription drug profile for the patient; accessing initial fulfillment information for fulfilling the prescription drug using the first claim adjudication entity; determining that one or more features associated with the prescription drug of the claim correspond to one or more criteria; in response to determining that one or more features correspond to the one or more criteria, transmitting a communication to a second server associated with a second claim adjudication entity, the communication identifying the prescription drug and requesting alternate fulfillment information; and selecting a claim adjudication process based on the initial fulfillment information and the alternate fulfillment information.

### CROSS-REFERENCE TO RELATED APPLICATION

This application is a non-provisional of and claims priority to U.S. Provisional Application No. 63/433,251, filed Dec. 16, 2022, which is hereby incorporated by reference in its entirety.

### BACKGROUND

Patients seek medical treatment in a variety of different ways. Some patients desire medical treatment in person by visiting a live medical professional. Other patients prefer to receive medical treatment virtually. Different systems are provided to access medical treatments based on the preferences of the patients. As part of treatment, patients are prescribed prescription drugs which need to be adjudicated by a specified entity.

### BRIEF DESCRIPTION OF THE DRAWINGS

FIG. 1 is a block diagram of an example first entity claim adjudication system, according to some embodiments.

FIG. 2 is an example database that may be deployed within the system of FIG. 1, according to some embodiments.

FIG. 3 is a block diagram of an example claim adjudication platform that may be deployed within the system of FIG. 1, according to some embodiments.

FIG. 4 is an example user interface of the first entity claim adjudication platform, according to example embodiments.

FIG. 5 is a flowchart illustrating example operations of the first entity claim adjudication platform, according to example embodiments.

FIG. 6 is a block diagram illustrating an example software architecture, which may be used in conjunction with various hardware architectures herein described.

FIG. 7 is a block diagram illustrating components of a machine, according to some example embodiments.

FIG. 8 is a functional block diagram of an example neural network that can be used for the inference engine or other functions (e.g., engines) as described herein to produce a predictive model.

### DETAILED DESCRIPTION

Example methods and systems for a claim adjudication platform are provided. Specifically, the methods and systems automatically select a claim adjudication process based on comparing initial fulfillment information of one adjudication server associated with a first entity with alternate fulfillment information of another adjudication server associated with a second entity. The first entity can be a claim adjudication entity that manages a prescription profile for a patient (e.g., such as an entity that is in an insurance plan network for the patient) and the second entity can be a discount drug company or discount pharmacy which may be outside of the insurance plan network for the patient. In the following description, for purposes of explanation, numerous specific details are set forth in order to provide a thorough understanding of example embodiments. It will be evident, however, to one of ordinary skill in the art that embodiments of the invention may be practiced without these specific details.

Patients seek medical treatment in a variety of different ways. Some patients desire medical treatment in person by visiting a live medical professional. Other patients prefer to receive medical treatment virtually. Different systems are provided to access medical treatments based on the preferences of the patients. After a patient visits a health care professional, the health care professional may recommend that the patient obtain prescription drugs (prescription medication) and creates a claim for the prescription drug. Prescription drugs can be obtained by the patient in a number of different ways and by having the claim processed through a variety of different claim adjudication entities. For example, the patient can visit a local pharmacy which is associated with or in the network of the patient's insurance plan as a first claim adjudication entity. As an alternative, the patient can obtain discount pharmacy coupons to redeem at an out-of-network pharmacy as a second claim adjudication entity to purchase the prescription drugs for cash without the benefit of the insurance plan.

To find the alternate or all the available claim adjudication options for processing the claim, the patient needs to navigate various information sources and pages of information and re-input various personal health information. Providing health information to a variety of sources to find all the available options for processing the claim for the prescription drug can expose personal health information to malicious sources and can compromise the patient's privacy and security. This also causes the patient to waste a great deal of time finding the various ways to fill a prescription by navigating through a multitude information sources which wastes system sources.

In addition, if the patient decides to obtain the prescription drugs from a claim adjudication entity that is out of the patient's network (e.g., is a claim adjudication entity that does not have access to the patient heath and prescription drug profile), the patient's health and safety can be put at risk. This is because the patient's network (e.g., the claim adjudication entity of the patient's health plan) can maintain the prescription drug profile and can evaluate risk of interaction between a newly prescribed drug and previously prescribed drugs and other health conditions. By obtaining the prescription drugs from a claim adjudication entity that is outside of the patient's health plan, the claim adjudication entity that maintains the patient's health records is unable to evaluate the health and safety risks of taking the new prescription drug. Finally, the patient may not get the benefit a having a deductible credited if the patient obtains the prescription drug from the claim adjudication entity that is out of the patient's network.

Sometimes, the out of network claim adjudication entity may have unreliable services which are inaccessible to the patient. This further frustrates the patient as the patient is unable to complete filling the prescription for the medication. The lack of resiliency and reliability makes such typical systems difficult to use and waste overall resources.

Typical systems fail to provide a single application that aggregates all of the different fulfillment information from the variety of claim adjudication entities (e.g., a variety of prescription drug sources that are in-network and out-of-network) into a single user interface. As a result, the health and safety of the patients are put at risk and patients are burdened with having to navigate multiple pages of information and repeatedly inputting their sensitive information to find all the possible sources from where a prescription drug can be obtained. This wastes a great deal of time and resources that can be devoted to other tasks.

The disclosed embodiments provide systems and methods that aggregate and collectively evaluate fulfillment information for a prescription drug claim from a variety of sources to automatically select a claim adjudication process for filling the prescription. Particularly, a single user interface or a single source of information, such as a claim adjudication entity that manages a prescription drug profile for a patient, can be accessed to find fulfillment information from each of a variety of different sources. An intelligent and automated evaluation of the fulfillment information from each of the sources is made in order to recommend or automatically adjudicate a claim for prescription drugs for a patient. Because the single user interface is provided to collectively analyze all the possible avenues for claim adjudication, having one claim adjudication entity fail at any given time does not result in loss of services to the patient as other avenues for adjudication are leveraged as backup.

In one example, the claim adjudication entity is a first claim adjudication entity that manages the prescription drug profile for the patient and the second claim adjudication entity is a discount prescription drug source. The first claim adjudication entity can compare costs and features of the prescription drug when obtained from the first claim adjudication entity with the costs and features of the same prescription drug when obtained from the second claim adjudication entity. If the costs and features are better when obtained from the second claim adjudication entity (e.g., the out-of-pocket cost for obtaining the prescription drug from the second claim adjudication entity is lower than the first claim adjudication entity), the first claim adjudication entity can select a claim adjudication process in which the prescription drugs are obtained using the second claim adjudication entity. The patient may be provided the prescription drugs at the cost available from the second claim adjudication entity without having to visit a physical location of the second claim adjudication entity and can have the deductible credited with the cost associated with the second claim adjudication entity. Also, the first claim adjudication entity can update the prescription drug profile to indicate that the patient has obtained the prescribed drugs even though the drugs were obtained from the out-of-network source.

By having the same entity that manages the prescription drug profile for the patient also perform the operations of automatically seeking fulfillment information form alternate claim adjudication entities, the prescription drug profile can be kept up to date with the prescription drugs a patient obtains and consumes even if such drugs are obtained from a claim adjudication entity that does not maintain or have access to the prescription drug profile. This also saves a great deal of time and resources because the user need not have to navigate through a multitude of pages of information to find alternate claim adjudication entities for filling a prescription and may not need to re-input of their medical information into unsecure sources of information.

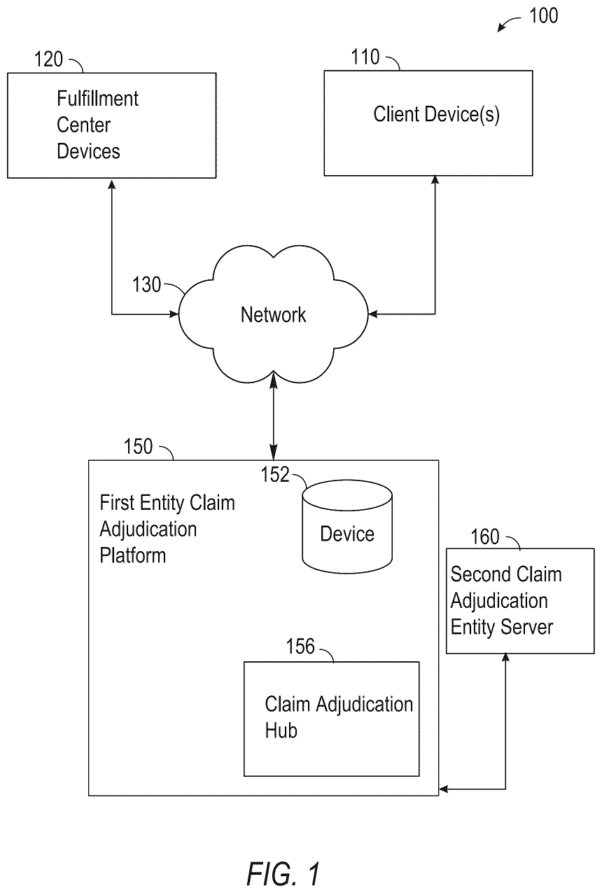

FIG. 1 is a block diagram showing an example claim adjudication system 100 according to various exemplary embodiments. The claim adjudication system 100 includes one or more client devices 110, one or more fulfillment center devices 120, a first entity claim adjudication platform 150, and second claim adjudication entity servers 160 (alternate claim adjudication servers) that are communicatively coupled over a network 130 (e.g., Internet, telephony network). Each of the one or more second claim adjudication entity servers 160 hosts an application or service that enables access to prescription drug features and costs from various associated fulfillment center devices 120.

For example, a given claim adjudication entity server one of the second claim adjudication entity servers 160 include a discount prescription drug provider that provides coupons to purchase drugs for cash (without using any insurance benefit) at a particular one of the fulfillment center devices 120. The adjudication platform 150 can manage a prescription drug profile for the patient and allow a patient to obtain prescription drugs at a cost determined by a health insurance plan of the patient, such as using the health insurance benefit).

The one or more second claim adjudication entity servers 160 can communicate with the first entity claim adjudication platform 150 to automatically provide alternate fulfillment information for a variety of prescription drugs, such as costs, discount coupons, quantity, and types of prescription drugs. The alternate fulfillment information can be provided by the second claim adjudication entity servers 160 in response to a communication transmitted by the 150 via the claim adjudication hub 156 requesting such information. Alternatively, alternate fulfillment information can be provided by the second claim adjudication entity servers 160 periodically as updates to the fulfillment information is generated by the second claim adjudication entity servers 160. In some cases, the patient interacts directly with the one or more second claim adjudication entity servers 160 to obtain the alternate fulfillment information. In some cases, the patient interacts with the one or more second claim adjudication entity servers 160 indirectly through an interface of the first entity claim adjudication platform 150.

The second claim adjudication entity servers 160 can, in some cases, be co-hosted on the adjudication platform 150. For example, a first server associated with a first of the alternate claim adjudication entities can be hosted on a shared cloud processing resource or web service that also hosts the servers of the first entity claim adjudication platform 150. This way, the amount of time it takes to send and receive communications between the servers of the different entities can be reduced. The shared cloud processing resource or web service can be associated with a third-party resource or service. In such cases, the adjudication platform 150 can request that the third-party resource reserve cloud processing resources in the same physical region for both the adjudication platform 150 and the first server associated with a first of the alternate claim adjudication entity. A second server associated with a second alternate claim adjudication entities can be hosted in its own region and outside of the adjudication platform 150. In such cases, the adjudication platform 150 can prioritize messages and inquires for fulfillment information sent to the first server over the second server.

As used herein, the term “client device” may refer to any machine that interfaces to a communications network (such as network 130) to access the first entity claim adjudication platform 150 and/or the second claim adjudication entity servers 160. The client device 110 may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network or the first entity claim adjudication platform 150.

In some cases, the first entity claim adjudication platform 150 is accessible over a global communication system, e.g., the Internet or world wide web. In such instances, the first entity claim adjudication platform 150 hosts a website that is accessible to the client devices 110. Upon accessing the website, the client devices 110 provide secure login credentials, which are used to access a profile associated with the login credentials and one or more patient profiles or patient information. As used herein, patient information and/or prescription drug profile includes any medical information associated with a patient including one or more prior medical insurance claims that were approved or denied, one or more electronic health records or medical health records, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, prescribed drugs or medication being taken by the patient, and/or one or more treatment preferences. One or more user interfaces associated with the first entity claim adjudication platform 150 are provided over the Internet via the website to the client devices 110.

Fulfillment center devices 120 can include the same or similar functionality as client devices 110 for accessing the first entity claim adjudication platform 150. In some cases, the fulfillment center devices 120 are used by “internal” users. Internal users are medical professionals, such as medical personnel, physicians, clinicians, healthcare providers, health-related coaches pharmacy benefit manager (PBM) operators, pharmacists, specialty pharmacy operators or pharmacists, or the like that are associated with, certified by, or employed by one or more organizations that provides the first entity claim adjudication platform 150. In some cases, the fulfillment center devices 120 are used by “external” users. External users are medical professionals and personnel, such as physicians, clinicians, and health-related coaches that are associated with or employed by a different (external) organization than that which provides the first entity claim adjudication platform 150. The PBM can be implemented as part of the adjudication platform 150.

The fulfillment center devices 120, in some cases, are used to access a respective server of the second claim adjudication entity servers 160. For example, the fulfillment center devices 120 can be a point-of-sale (POS) device at a local pharmacy that is configured to receive fulfillment information (e.g., drug costs, types, and features) from the adjudication platform 150 and/or the second claim adjudication entity servers 160. Based on the received fulfillment information, the POS device authorizes the release and provision of the prescription drugs to a patient who is vising the fulfillment center devices 120. The POS device of the fulfillment center devices 120 can charge the patient for any out-of-pocket costs for obtaining the prescription drugs and can also receive payment, if any, from a health insurance plan associated with the patient.

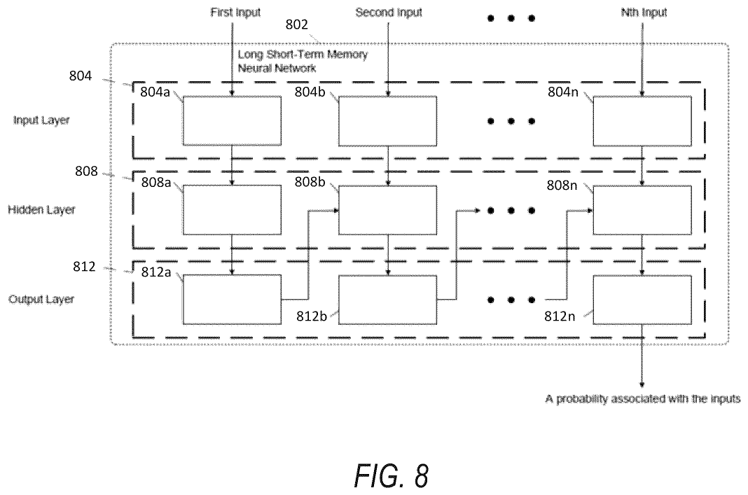

In some examples, the first entity claim adjudication platform 150 (and specifically the claim adjudication hub 156) can implement a machine learning technique or machine learning model, such as a neural network (discussed below in connection with FIG. 8). The machine learning model can be trained to establish a relationship between a plurality of prescription drug features and likelihoods indicating that a set of alternate fulfillment information of the second server outranks a set of fulfillment information of the first server. The machine learning model can then receive a new set of prescription drug features for a prescribed drug and can estimate or predict a likelihood that an alternate claim adjudication server will outrank or provide an improve benefit for fulfilling the prescription drug over the claim adjudication server of an entity that manages the prescription drug profile for the patient. The machine learning model can condition the communication with the alternate adjudication server on the basis of the likelihood that processing the claim with the alternate claim adjudication server will benefit the patient over processing the claim with the claim adjudication server of the entity that manages the prescription drug profile. This reduces the number of times communication between the adjudication platform 150 is made with the second claim adjudication entity servers 160 which reduces network congestion.

In an example, the machine learning model can be trained by obtaining a batch of training data comprising a first set of the plurality of training prescription drug features and ground truth likelihoods indicating that the set of alternate fulfillment information of the second server outranks the set of fulfillment information of the first server. The machine learning model processes the first set of the plurality of training prescription drug features by the machine learning model to generate an estimated rank. The machine learning model computes a loss based on a deviation between the estimated rank and the ground truth likelihoods and updates one or more parameters of the machine learning model based on the computed loss. These training operations can be repeated for multiple batches of training data and/or until a stopping criterion is reached.

In some examples, the first entity claim adjudication platform 150 can condition the communication with the alternate adjudication server(s) on the basis of various criteria in addition to or alternative to the likelihood predicted by the machine learning model. For example, the first entity claim adjudication platform 150 can condition the communication based on a class associated with the prescription drug, popularity of the prescription drug, a manufacturer associated with the prescription drug, current supply shortage information associated with the prescription drug, region associated with the first entity claim adjudication platform 150 (or patient who needs the prescription drug), and/or region associated with the alternate adjudication server(s). For example, the first entity claim adjudication platform 150 may receive a request to adjudicate a claim for an individual prescription drug. The first entity claim adjudication platform 150 may determine that the individual prescription drug is in short supply as the various criteria. In such cases, the first entity claim adjudication platform 150 may avoid or prevent communicating with the alternate adjudication server(s) because there is a very low likelihood that the alternate adjudication server(s) will have the individual prescription drug in stock. As another example, the first entity claim adjudication platform 150 may receive a request to adjudicate a claim for an individual prescription drug that is associated with popularity that transgresses a threshold. In such cases, the first entity claim adjudication platform 150 may avoid or prevent communicating with the alternate adjudication server(s) because there is a very low likelihood that the alternate adjudication server(s) will have the individual prescription drug in stock or the first entity claim adjudication platform 150 may be better suited to fulfill the individual prescription drug prescription given its popularity.

Similarly, the first entity claim adjudication platform 150 may determine that the individual prescription drug is in short supply as the various criteria. In such cases, the first entity claim adjudication platform 150 may increase a priority for messages sent to the alternate adjudication server(s) because of the need to find an entity that has the individual prescription drug in stock. So rather than preventing communicating with the alternate adjudication server(s), the first entity claim adjudication platform 150 may prioritize a request or inquiry associated with the individual drug that is in low supply over another inquire associated with another prescription drug that is not in low supply. The first entity claim adjudication platform 150 may receive a request to adjudicate a claim for an individual prescription drug that is associated with popularity that transgresses a threshold. In such cases, the first entity claim adjudication platform 150 increase a priority for messages sent to the alternate adjudication server(s) because of the need to find an entity that has the individual prescription drug.

In some examples, the first entity claim adjudication platform 150 may collect fulfillment information from multiple alternate adjudication servers. The first entity claim adjudication platform 150 can aggregate and compare all of the various fulfillment information collected from the multiple alternate adjudication servers. The first entity claim adjudication platform 150 can then select an individual alternate adjudication server from the multiple alternate adjudication servers based on the aggregation and comparison. For example, the first entity claim adjudication platform 150 can receive price information from each of the multiple alternate adjudication servers and can select one of the multiple alternate adjudication servers that provides a lowest price for the same prescription drug to fulfill the prescription for a patient.

In some examples, the adjudication platform 150 communicates directly with the second claim adjudication entity servers 160. In some examples, the adjudication platform 150 communicates with the second claim adjudication entity servers 160 via the claim adjudication hub 156 and/or a firewall. The adjudication platform 150 (which includes a first server of the first claim adjudication entity) receives a claim for a prescription drug for a patient. The first claim adjudication entity manages a prescription drug profile for the patient. The adjudication platform 150 accesses initial fulfillment information for fulfilling the prescription drug using the first claim adjudication entity and determines that one or more features associated with the prescription drug of the claim correspond to one or more criteria. The one or more criteria can include a minimum or maximum price, a class of drug, popularity of the drug, supply status of the drug (whether the drug is in short supply), manufacturer of the drug, and/or region information associated with the drug.

The adjudication platform 150, in response to determining that one or more features associated with the prescription drug of the claim correspond to the one or more criteria, transmits a communication to a second server associated with a second claim adjudication entity (e.g., a given one of the second claim adjudication entity servers 160). The communication identifies the prescription drug and requesting alternate fulfillment information. The adjudication platform 150 selects a claim adjudication process based on the initial fulfillment information and the alternate fulfillment information. For example, the adjudication platform 150 selects whether the fulfillment center devices 120 fulfills the prescription using a benefit associated with the adjudication platform 150 and/or a benefit or cost associated with the given one of the second claim adjudication entity servers 160.

In some examples, the adjudication platform 150 can condition communicating with one or more of the second claim adjudication entity servers 160 based on load information that is dynamically and/or statically determined. For example, the second claim adjudication entity servers 160 can include a first server associated with a first alternate claim adjudication entity and a second server associated with a second alternate claim adjudication entity. The adjudication platform 150 can determine that, based on historical information, the first server provides fulfillment information that outranks fulfillment information provided by the second server for the same prescription drug or class of prescription drugs. The adjudication platform 150 can receive a request to fulfill a prescription for an individual prescription drug. The adjudication platform 150 can determine, based on historical information, that, at the current day, season, region, and/or time, the first server is currently overloaded or has a load that causes responses to be processed in an amount of time that transgresses a threshold. In such cases, the adjudication platform 150 can transmit an inquire to the second server instead of the first server given the overloading of the first server at the current time even though the fulfillment information that is provided by the second server is associated with a lower rank than that which would be provided by the first server.

In some examples, the adjudication platform 150 determines that the one or more features correspond to the one or more criteria by receiving input from the patient for opting into an alternate claim adjudication process including communication with the second server adjudication entity. In some implementations, the adjudication platform 150 causes presentation, on client device 110, of adjudication entities including at least the second claim adjudication entity. The adjudication platform 150 receives, from the client device 110, a selection adjudication entities that are on the list. In some examples, the adjudication platform 150, in response to determining that one or more features (e.g., costs) associated with the prescription drug correspond to the one or more criteria (e.g., threshold cost), transmits a plurality of communications to each server of the subset of the alternate adjudication entities indicated in the selection received from the client device 110. Each of the communications can identify the prescription drug and requests alternate fulfillment information.

In some examples, the adjudication platform 150 determines that the one or more features correspond to the one or more criteria by applying a model to the one or more features associated with the prescription drug to rank the prescription drug. The adjudication platform 150 determines that the rank transgresses a threshold rank. In some implementations, the model includes a machine learning model including a neural network. The machine learning model can be trained to establish a relationship between a plurality of prescription drug features and likelihoods indicating that a set of alternate fulfillment information of the second server outranks a set of fulfillment information of the first server.

In some examples, the machine learning model is trained by performing operations including: obtaining a batch of training data including a first set of the plurality of training prescription drug features and ground truth likelihoods indicating that the set of alternate fulfillment information of the second server outranks the set of fulfillment information of the first server; processing the first set of the plurality of training prescription drug features by the machine learning model to generate an estimated rank; computing a loss based on a deviation between the estimated rank and the ground truth likelihoods; and updating one or more parameters of the machine learning model based on the computed loss.

In some examples, the adjudication platform 150 determines that the one or more features correspond to the one or more criteria by accessing a predetermined list adjudication entity. The adjudication platform 150 determines that an identifier (e.g., name, quantity, and type) represented by the one or more features of the prescription drug matches one or more identifiers on the predetermined list of prescription drugs. In some examples, the adjudication platform 150 initializes a timer in response to transmitting the communication to the second server of a first alternate adjudication entity. In some implementations, the adjudication platform 150 determines that a response from the second server including the alternate fulfillment information has been received prior to expiration of the timer. In such cases the adjudication platform 150 selects the claim adjudication process based on the initial fulfillment information and the alternate fulfillment information in response to determining that the response has been received prior to the expiration of the timer.

In some examples, the adjudication platform 150 can provide or use different timers for different servers of the second claim adjudication entity servers 160. In such cases, the adjudication platform 150 can determine whether a first server response to inquiries within a first timer period. If the first server responds within the first timer period to provide fulfillment information, the adjudication platform 150 can continue to send requests for fulfillment information to the first server. If not, the adjudication platform 150 can temporarily or permanently send requests for fulfillment information to a second server associated with a different alternate adjudication entity. The adjudication platform 150 can determine whether the second server response to inquiries within a second timer period (different from the first timer period). If the second server responds within the second timer period to provide fulfillment information, the adjudication platform 150 can continue to send requests for fulfillment information to the second server. If not, the adjudication platform 150 can temporarily or permanently send requests for fulfillment information to a third server associated with another alternate adjudication entity and/or back to the first server.

In some examples, the adjudication platform 150 can prevent sending inquires to certain servers of the second claim adjudication entity servers 160 based on time of day. For example, the adjudication platform 150 can prevent sending any inquiries to any of the second claim adjudication entity servers 160 between 2-4 PM on weekdays and 12-5 PM on weekends. During those times, the adjudication platform 150 can adjudicate claims for prescription drugs and prevents sending requests to the second claim adjudication entity servers 160. When the current time is outside of those periods of time, the adjudication platform 150 can send the inquires to the second claim adjudication entity servers 160 based on one or more criteria, as discussed above and below.

In some examples, the adjudication platform 150 determines that a response from the second server including the alternate fulfillment information has failed to be received prior to expiration of the timer. In such cases, the adjudication platform 150 selects the initial fulfillment information in response to determining that the response has failed to be received prior to the expiration of the timer and/or selects another server associated with a second alternate adjudication entity.

In some examples, the adjudication platform 150 stores a history of response times for each server of the second claim adjudication entity servers 160. Namely, the adjudication platform 150 can store a table that maps prescription drugs and/or classes of prescription drugs to alternate servers of the second claim adjudication entity servers 160 and to times or day when the requests are sent. Each time an individual server of the second claim adjudication entity servers 160 is queried to provide fulfillment information, the adjudication platform 150 measures how long between the time the inquiry is sent to the individual server and when a response is received and updates the amount of time that is stored of the individual server in the table for the time of day. This allows the adjudication platform 150 to determine how long different ones of the second claim adjudication entity servers 160 take to process inquiries for fulfilment information on a per drug or per class of drug basis on different times of the day. Using this historical information, the adjudication platform 150 can select one of the second claim adjudication entity servers 160 to process a claim for a prescription drug that will return the fulfillment information the fastest or within a threshold period of time.

In some examples, the adjudication platform 150 can send a request for fulfillment information for an individual prescription drug to an individual server of the second claim adjudication entity servers 160. The adjudication platform 150 can receive a response to the inquire from the individual server. The adjudication platform 150 can compare the amount of time the individual server took to generate and send the response to the amount of time stored for the individual server in the table. If the amount of time is determined to be exponentially longer than the stored amount of time and/or if the amount of time is exponentially longer than a specified timer period, the adjudication platform 150 can select another server associated with a different one of the second claim adjudication entity servers 160 to process subsequent inquiries and generate fulfillment information instead of sending additional inquiries to the same individual server.

In some examples, after a threshold period of time (e.g., after 2 or 3 hours) elapses from when the adjudication platform 150 determined that the amount of time it took the individual server of the second claim adjudication entity servers 160 to generate a response to the query for the individual prescription drug was exponentially longer than the stored amount of time and/or exponentially longer than the specified timer period, the adjudication platform 150 can generate and send a test message for transmission to the individual server. The test message can include a test inquiry for fulfillment information for a test prescription drug. The adjudication platform 150 can compute a current amount of time representing how long the individual server takes to respond to the test inquire with the fulfillment information. The adjudication platform 150 can determine whether the current amount of time with the historical amount of time stored of the individual server in the table and/or the threshold. If the adjudication platform 150 determines that the current amount of time is within the historical amount of time stored of the individual server in the table and/or the threshold, the adjudication platform 150 can resume transmitting subsequent requests for fulfillment information to the individual server of the second claim adjudication entity servers 160.

In some examples, the adjudication platform 150 identifies one or more peak times for communicating with the second server. The adjudication platform 150 determines that a current time falls within the one or more peak times and prevents transmitting the communication to the second server in response to determining that the current time falls within the one or more peak times. In some examples, the adjudication platform 150 compares the initial fulfillment information with the alternate fulfillment information and determines that the alternate fulfillment information is associated with a higher priority than the initial fulfillment information (e.g., the alternate fulfillment information is associated with a lower cost than the initial fulfillment information). In response, to the adjudication platform 150 selects, as the claim adjudication process, operations for adjudicating the claim for the prescription drug via the second claim adjudication entity.

In some examples, the adjudication platform 150 utilizes (updates) the prescription drug profile for the patient by the first claim adjudication entity in response to adjudicating the claim for the prescription drug via the second claim adjudication entity. In some examples, the adjudication platform 150 stores an identification of the prescription drug in the prescription drug profile for the patient and determines drug interaction information between the prescription drug and other drugs listed in the prescription drug profile.

In some examples, the adjudication platform 150 transmits the communication from the first server to the second server via an intermediate hub, such as claim adjudication hub 156. In some aspects, the prescription drug profile includes at least one information for the patient, patient health information, past medical recommendations, past treatment recommendations, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, or one or more treatment preferences. In some examples, the adjudication platform 150 receives the alternate fulfillment information from the second server. The adjudication platform 150 modifies the initial fulfillment information based on the alternate fulfillment information.

The network 130 may include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The fulfillment center devices 120 can be used to access pharmacy claims, medical data (e.g., a prescription drug profile 230 stored in database 152), laboratory data and the like for one or more patients that the fulfillment center devices 120 are authorized to view. This drug fulfillment information 210 can be maintained in a database 152 by the first entity claim adjudication platform 150 or in a third-party database accessible to the first entity claim adjudication platform 150 and/or the fulfillment center devices 120. The drug fulfillment information 210 can list a variety of different prescription drug features (e.g., names, types, quantities, and/or costs) and a variety of different claim adjudication entities that provide the prescription drugs with the specified drug features. For example, the drug fulfillment information 210 can store initial fulfillment information that specifies a first cost for a first prescription drug when fulfilled by a first adjudication entity and can store alternative fulfillment information that specifies a second cost for the first prescription drug when fulfilled by a second adjudication entity, such as via one of the second claim adjudication entity servers 160. While the disclosed examples are discussed in relation to cost as the fulfillment information, any other type of fulfillment information can be similarly used for performing the disclosed functions.

In some embodiments, the client devices 110 and the first entity claim adjudication platform 150 can be communicatively coupled via an audio call (e.g., VoIP, Public Switched Telephone Network, cellular communication network, etc.) or via electronic messages (e.g., online chat, instant messaging, text messaging, email, and the like). While FIG. 1 illustrates a single client device 110 and a single healthcare provider device 120, it is understood that a plurality of such devices can be included in the system 100 in other embodiments. As used herein, the term “client device” may refer to any machine that interfaces to a communications network (such as network 130) to obtain resources from one or more server systems or other client devices.

In some embodiments, the first entity claim adjudication platform 150 includes the claim adjudication hub 156. In some examples, the claim adjudication hub 156 and/or adjudication platform 150 processes medical information input by a patient via the client device 110 and/or patient information stored in one or more databases. For example, the client device 110 can present a graphical user interface to the patient. The graphical user interface can receive input from the patient that provides a variety of medical information, such as patient health information, patient demographic information, patient in-network insurance coverage, patient out-of-network insurance coverage, patient location, and/or one or more treatment preferences. The input can also identify storage locations of one or more electronic health records and/or claims information.

As referred to here, the “first adjudication entity” is an entity that manages a prescription drug profile for a patient and is in a network of an health or medical insurance plan for the patient. As referred to here, the “alternative adjudication entity” or “second adjudication entity” is an entity that is outside of a health or medical insurance plan for the patient and does not have access to the prescription drug profile for the patient, such as a discount pharmacy. Sometimes the first adjudication entity can become the second adjudication entity and vice versa. The input can also specify preferences for a variety of alternative adjudication entities. This input can be stored as part of prescription drug profile 230 associated with the patient in the adjudication platform 150. The input can also specify criteria that is used to trigger processing a claim by an alternate adjudication entity instead of the first entity claim adjudication platform 150. For example, the criteria can include an indication of a threshold price difference between a cost of the prescription drug fulfilled by the first adjudication entity relative to the cost for fulfilling the same prescription drug by the second adjudication entity. If the difference in cost transgresses the threshold, the adjudication platform 150 automatically fulfills the prescription drug claim using the second adjudication entity.

As another example, the criteria can include a deductible minimum. For example, if the patient's deductible has not been met or is below a certain threshold value, then the adjudication platform 150 can process the prescription drug claim using the second adjudication entity instead of the first adjudication entity if other criteria is met, such as if the drug is cheaper through the second adjudication entity. If the patient's deductible has been met or is currently above the certain threshold value, then the adjudication platform 150 can process the prescription drug claim using the first adjudication entity instead of the second adjudication entity even if the drug is cheaper through the second adjudication entity. The satisfaction of the criteria can trigger communication between the adjudication platform 150 and one or more of the second claim adjudication entity servers 160 to determine whether the prescription drug is more economical or cheaper through the second adjudication entity.

In some cases, the patient health information can be input using the client device 110 by receiving selection from the patient of one or more checkboxes from a user interface, each associated with a different medical condition, such as high blood pressure, diabetes, obesity, demographics, and so forth. The patient in-network insurance coverage and out-of-network insurance coverage be input by receiving selection from the patient of one or more checkboxes from a user interface, each associated with a different health insurance carrier. Based on the selected health insurance carrier and health plan information input by the patient, the patient in-network and out-of-network insurance coverages can be automatically determined and retrieved. The patient location can be automatically determined by accessing location information (e.g., GPS coordinates) from the client device 110 and/or by requesting a residential address from the patient. The treatment preferences can be input by receiving selection from the patient specifying how the patient likes to receive treatment, such as in-person, virtually, in a structured format, in aspirational format (e.g., a format that lacks structure), and so forth.

The claim adjudication hub 156 can store a previously trained machine learning model. The machine learning model can process the information input by the patient and/or by the fulfillment center devices 120 that identifies a prescription drug. The claim adjudication hub 156 can conditionally communicate with the second claim adjudication entity servers 160 based on determining whether the prescription drug satisfies one or more criteria, such as in response to an output of the machine learning model and/or based on previously provided criteria selected by the patient using the client device 110. For example, if a likelihood or rank output by the machine learning model transgresses a threshold, the claim adjudication hub 156 communicates with the second claim adjudication entity servers 160 to obtain alternate fulfillment information.

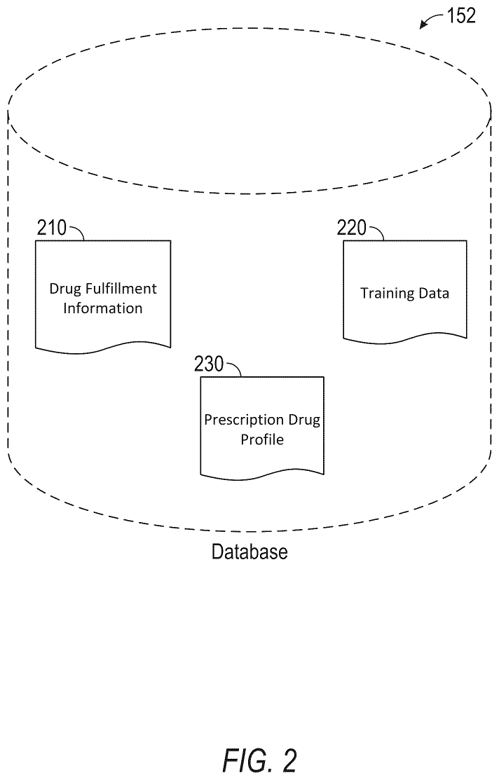

FIG. 2 is an example database 152 that may be deployed within the system of FIG. 1, according to some embodiments. The database 152 includes drug fulfillment information 210, prescription drug profile 230, and training data 220. The drug fulfillment information 210 can be generated or accessed by the first entity claim adjudication platform 150 and can be updated based on information received from one or more second claim adjudication entity servers 160.

For example, the first entity claim adjudication platform 150 can access one or more patient records from one or more sources, including pharmacy claims, benefit information, prescribing physician information, dispensing information (e.g., where and how the patient obtains their current medications), demographic information, prescription information including dose quantity and interval, and input from a patient received via a user interface presented on the client device 110 and so forth. The first entity claim adjudication platform 150 can collect this information from the patient records and generates a patient features vector that includes this information, such as the prescription drug profile 230.

The adjudication platform 150 can, in response to receiving a new prescription drug claim, access the prescription drug profile 230 for the patient. The adjudication platform 150 can determine based on the prescription drug profile 230 whether the adjudication platform 150 has been authorized by the patient to communicate with alternative adjudication entities to obtain prescription drugs. The adjudication platform 150 evaluates one or more criteria associated with the prescription drug to condition the communication with the second claim adjudication entity servers 160 to obtain alternative fulfillment information. For example, the adjudication platform 150 can determine if the prescription drug is on a list of drugs available for fulfillment by one or more alternative fulfillment entities, such as by comparing a name and quantity of the drug with a predetermined list of names and quantities of drugs. The predetermined list can also specify the second claim adjudication entity associated with the drug on the list from which the drug can be obtained. If the prescribed drug matches one of the drugs on the predetermined list, the adjudication platform 150 can transmit the communication to the second claim adjudication entity servers 160, such as to the server of the entity that is on the list that matches the drug. As another example, the adjudication platform 150 can access one or more peak times for communicating with the second claim adjudication entity servers 160. The adjudication platform 150 can condition transmitting the communication on whether the current time is within the peak times or not. This can reduce network congestion and traffic by only transmitting the communication to the second claim adjudication entity servers 160 if the current time is outside of the one or more peak times.

In some cases, the adjudication platform 150 can determine that a given one of the second claim adjudication entity servers 160 is down or fails to respond within a specified interval of time. In such cases, the adjudication platform 150 can prevent transmitting the communication to the given one of the second claim adjudication entity servers 160. Instead, the adjudication platform 150 can access a history of prior information received from the second claim adjudication entity servers 160 to estimate the fulfillment information. This reduces network congestion and allows the given one of the second claim adjudication entity servers 160 additional time to resume services. In some cases, the adjudication platform 150 throttles the quantity of communications that are transmitted to the given one of the second claim adjudication entity servers 160 so as to not overwhelm the adjudication entity. Namely, if the given one of the second claim adjudication entity servers 160 was determined to be down or unavailable at a particular time, then at a threshold period of time later (e.g., 2 hours later), the adjudication platform 150 transmits a first quantity of communications (e.g., 10 percent of the total needed fulfillment information) requesting fulfillment information that is less than a total quantity of needed fulfillment information for the second entity. After a period of time later than the threshold period of time (e.g., 1 day later), the adjudication platform 150 transmits a second quantity of communications which can be approximately 50 percent of the total needed fulfillment information from the second entity.

The adjudication platform 150 can determine that responses from the given one of the second claim adjudication entity servers 160 are received in less than a threshold period of time. In response, the adjudication platform 150 resumes transmission of each of a plurality of communications corresponding to 100% of the total needed fulfillment information from the second entity.

The adjudication platform 150 can access the drug fulfillment information 210 to obtain initial fulfillment information for the prescription drug claim. This can indicate the cost, such as a deductible or copay amount, the patient needs to pay to obtain the prescription drug using the first adjudication entity. The adjudication platform 150 can also determine if the drug fulfillment information 210 indicates alternative fulfillment information for obtaining the drug from alternative adjudication entities. If the alternative fulfillment information is present in the drug fulfillment information 210, the adjudication platform 150 compares the initial fulfillment information with the available alternative fulfillment information. For example, the adjudication platform 150 compares the cost for obtaining the drug using the first adjudication entity with the cost for obtaining the drug using the second adjudication entity. If the adjudication platform 150 determines that the alternative fulfillment information is better for the patient (e.g., the cost for obtaining the drug via the second adjudication entity is lower than the cost for obtaining the drug using the first adjudication entity), the automatically instructs the fulfillment center devices 120 to fill the prescription for the drug using the second adjudication entity. The adjudication platform 150 can communicate an indication of the drug that is being fulfilled to the corresponding server of the second claim adjudication entity servers 160 associated with the second adjudication entity.

In some cases, the adjudication platform 150 determines that there is no alternative fulfillment information stored in the drug fulfillment information 210. In such cases, the adjudication platform 150 initializes a timer (e.g., 25 second timer) for obtaining the alternative fulfillment information from one or more of the second claim adjudication entity servers 160. The adjudication platform 150 transmits a communication to the second claim adjudication entity servers 160 that requests the alternative fulfillment information for the prescription drug claim. The communication can identify the type and quantity of the prescription drug. If the adjudication platform 150 receives the fulfillment information before the timer expires (e.g., reaches 25 seconds), the adjudication platform 150 compares the received alternative fulfillment information from each of the second claim adjudication entity servers 160 with the initial fulfillment information. If the adjudication platform 150 determines that the alternative fulfillment information is better for the patient (e.g., the cost for obtaining the drug via the second adjudication entity is lower than the cost for obtaining the drug using the first adjudication entity), the automatically instructs the fulfillment center devices 120 to fill the prescription for the drug using a corresponding one of the adjudication entities of the second claim adjudication entity servers 160.

If the adjudication platform 150 receives the alternative fulfillment information after the timer expires (e.g., reaches 25 seconds) (e.g., fails to receive the alternative fulfillment information before expiration of the timer), the adjudication platform 150 automatically processes the prescription drug claim using the first adjudication entity. The adjudication platform 150 can store the alternative fulfillment information received after expiration of the timer in the drug fulfillment information 210 after processing the prescription drug claim for the patient and can use this information to process future drug claims for this same prescription drug.

The adjudication platform 150 can update the prescription drug profile 230 to identify the prescription drug that has been filled by the patient. The adjudication platform 150 can update the prescription drug profile 230 with such information even when the second adjudication entity is used to fill the prescription. The adjudication platform 150 can also analyze other prescription drugs stored in the prescription drug profile 230 to determine health and safety information, such as whether the prescribed drug can be taken with other medication currently being taken by the patient. The adjudication platform 150 can also determine drug interaction information between the drug obtained from the second adjudication entity and future drugs prescribed to the patient.

The training data 220 includes training sets of a plurality of training prescription drug features and ground truth likelihoods indicating that the set of alternate fulfillment information of the second server (of the second adjudication entity) outranks the set of fulfillment information of the first server (of the first adjudication entity). The training data 220 is used to train a machine learning model implemented by the service of claim adjudication hub 156 to generate estimates of ranks for a prescription drug, such as indicating of a likelihood that the second adjudication entity provides the drug for a cheaper cost than the first adjudication entity.

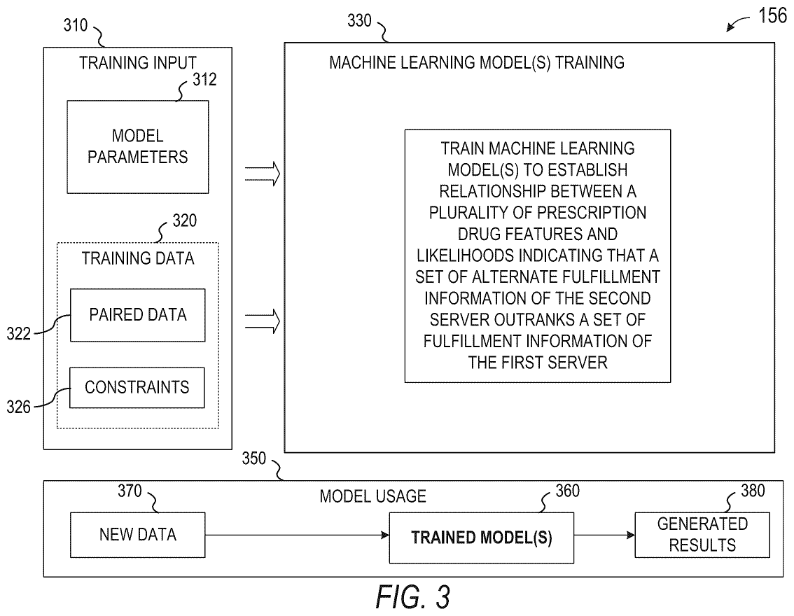

FIG. 3 is a block diagram of an example service of claim adjudication hub 156 that may be deployed within the system of FIG. 1, according to some embodiments. Training input 310 includes model parameters 312 and training data 320 (e.g., training data 220 (FIG. 2)) which may include paired training data sets 322 (e.g., input-output training pairs) and constraints 326. Model parameters 312 stores or provides the parameters or coefficients of corresponding ones of machine learning models. During training, these parameters 312 are adapted based on the input-output training pairs of the training data sets 322. After the parameters 312 are adapted (after training), the parameters are used by trained models 360 to implement the trained machine learning models on a new set of data 370.

Training data 320 includes constraints 326 which may define the constraints of a given patient information features. The paired training data sets 322 may include sets of input-output pairs, such as pairs of a plurality of training patient information features and types of medical tests performed based on visits to various types of medical professionals associated with the training patient information features. Some components of training input 310 may be stored separately at a different off-site facility or facilities than other components.

Machine learning model(s) training 330 trains one or more machine learning techniques based on the sets of input-output pairs of paired training data sets 322. For example, the model training 330 may train the machine learning (ML) model parameters 312 by minimizing a loss function based on one or more ground-truth type of service of care. The ML model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

In one example, the ML model receives a batch of training data that includes a first set of the plurality of training prescription drug features and ground truth likelihoods indicating that the set of alternate fulfillment information of the second server outranks the set of fulfillment information of the first server. The ML model generates a feature vector based on the first set of the plurality of training patient information features and generates an estimated set of ranks for the prescription drug features. The prediction is compared with the ground truth likelihoods and one or more parameters of the ML model are updated based on the comparison.

The result of minimizing the loss function for multiple sets of training data trains, adapts, or optimizes the model parameters 312 of the corresponding ML models. In this way, the ML model is trained to establish a relationship between a plurality of prescription drug features and likelihoods indicating that a set of alternate fulfillment information of the second server outranks a set of fulfillment information of the first server.

After the machine learning model is trained, new data 370, including one or more prescription drug features are received. The trained machine learning technique may be applied to the new data 370 to generate results 380 including a prediction of likelihood that a second adjudication entity has better fulfillment information than the first adjudication entity.

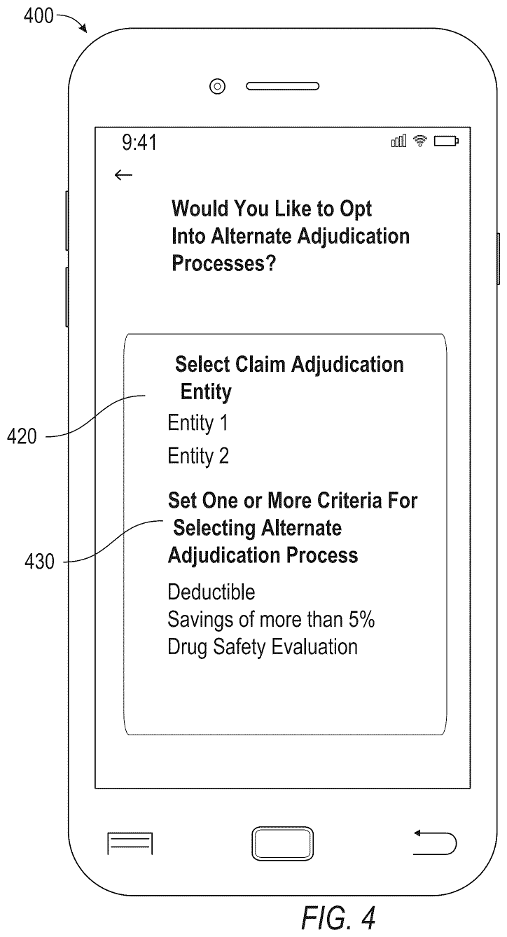

FIG. 4 is an example user interface 400 of the claim adjudication system 100, according to some examples. For example, a client device 110 can present a user interface that allows a patient to participate in alternate adjudication options or processes for processing a prescription drug claim. The interactions and selections made via the user interface 400 can be stored in the prescription drug profile 230 for the patient. For example, the user interface 400 can present a list of different alternate claim adjudication entities in a region 420. User input is received by the client device 110 that selects one or more of the claim adjudication entities listed in the region 420. The adjudication platform 150 can condition the transmission of the communication to the second claim adjudication entity servers 160, to obtain alternate fulfillment information for a prescription drug claim for the patient, to only those entities that are selected by the user from region 420.

The user interface 400 can also include a criteria selection region 430. The user interface 400 can receive input from the user that specifies one or more criteria to use to condition the transmission of the communication to the second claim adjudication entity servers 160, to obtain alternate fulfillment information for a prescription drug claim for the patient, only when the one or more criteria specified in the criteria selection region 430 are satisfied. The conditions can specify deductible options that indicate the amount remaining on the deductible that is used to condition transmission of the communication to the second claim adjudication entity servers 160. Namely, the deductible can specify that if the deductible is met or within a threshold amount, then the communication with the second claim adjudication entity servers 160 is not performed and the prescription drug claim is to be processed by the adjudication platform 150 through the medical health plan of the patient. The criteria specified in the criteria selection region 430 can indicate a threshold price difference (e.g., how much cost savings are provided to the patient) between filling the prescription with the first adjudication entity versus the second adjudication entity. For example, if the first adjudication entity can provide the drug for less than 5% more cost than the second adjudication entity, the patient can request to have the drug filled by the first adjudication entity even though the drug is cheaper when filled by the second adjudication entity.

The criteria can specify whether the patient would like a drug safety evaluation. If this option is selected, then a drug safety check is performed when filling the prescription through the second adjudication entity. Also, the drug filled by the second adjudication entity is updated in the prescription drug profile 230 for the patient.

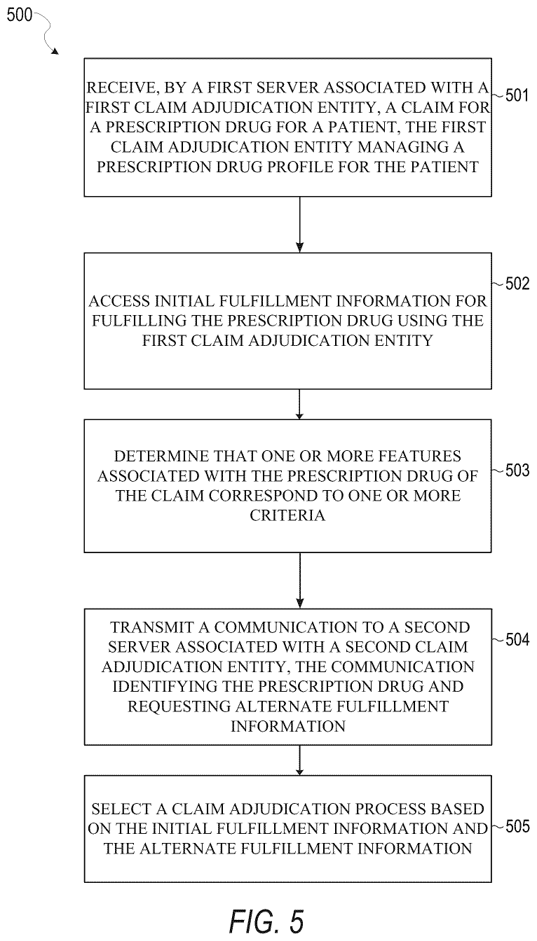

FIG. 5 is a flowchart illustrating example operations of the first entity claim adjudication platform in performing a method or process 500, according to example embodiments. The process 500 may be embodied in computer-readable instructions for execution by one or more processors such that the operations of the process 500 may be performed in part or in whole by the functional components of the system 100; accordingly, the process 500 is described below by way of example with reference thereto. However, in other embodiments, at least some of the operations of the process 500 may be deployed on various other hardware configurations. Some or all of the operations of process 500 can be in parallel, out of order, or entirely omitted.

At operation 501, the system 100 receives, by a first server associated with a first claim adjudication entity, a claim for a prescription drug for a patient, the first claim adjudication entity managing a prescription drug profile for the patient, as discussed above.

At operation 502, the system 100 accesses initial fulfillment information for fulfilling the prescription drug using the first claim adjudication entity, as discussed above.

At operation 503, the system 100 determines that one or more features associated with the prescription drug of the claim correspond to one or more criteria, as discussed above.

At operation 504, the system 100, in response to determining that one or more features associated with the prescription drug of the claim correspond to the one or more criteria, transmits a communication to a second server associated with a second claim adjudication entity, the communication identifying the prescription drug and requesting alternate fulfillment information, as discussed above.

At operation 505, the system 100 selects a claim adjudication process based on the initial fulfillment information and the alternate fulfillment information, as discussed above.

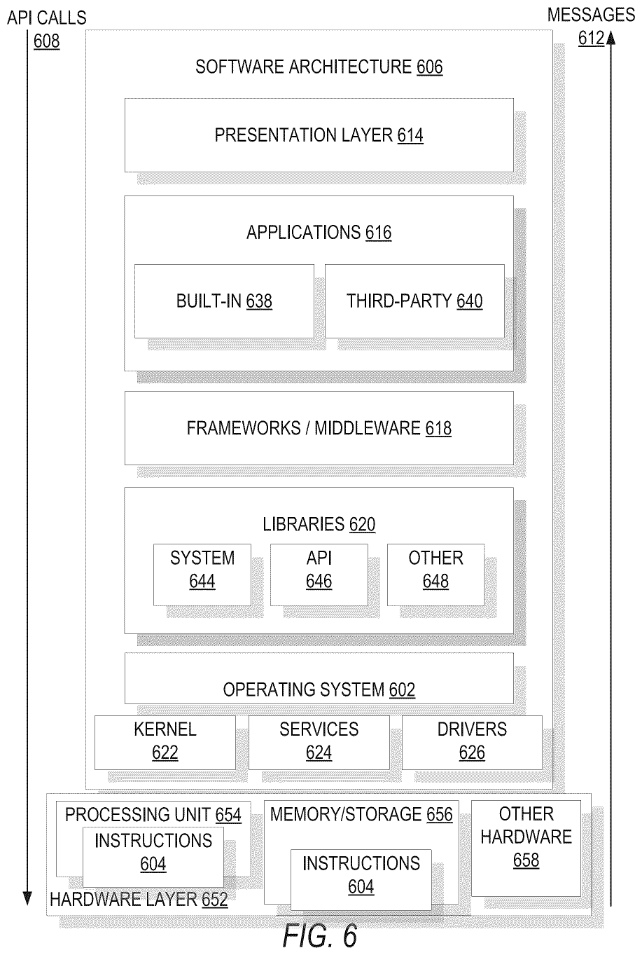

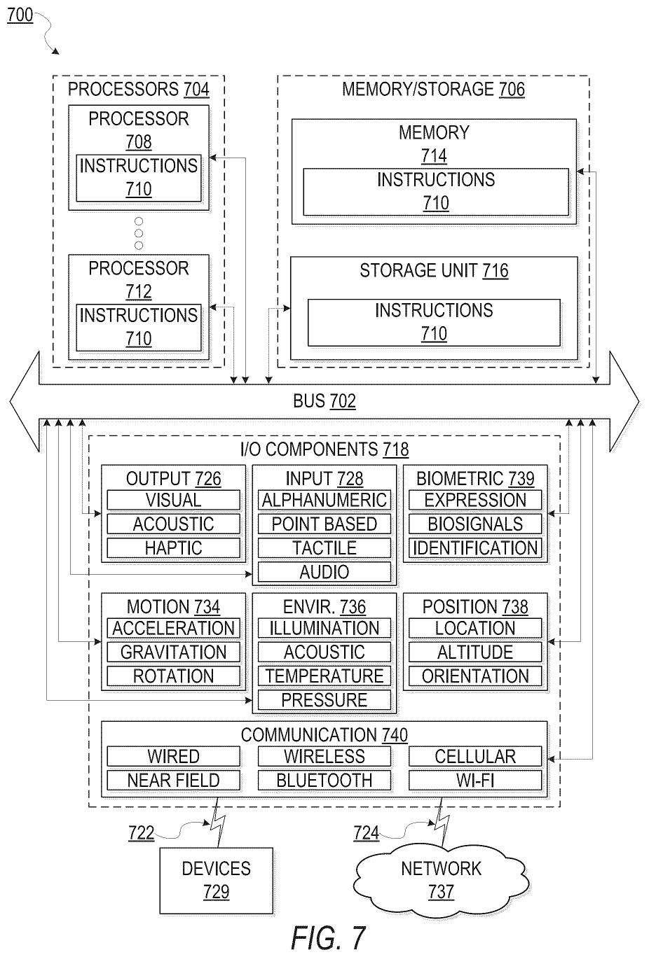

FIG. 6 is a block diagram illustrating an example software architecture 606, which may be used in conjunction with various hardware architectures herein described. FIG. 6 is a non-limiting example of a software architecture and it will be appreciated that many other architectures may be implemented to facilitate the functionality described herein. The software architecture 606 may execute on hardware such as machine 700 of FIG. 7 that includes, among other things, processors 704, memory 714, and input/output (I/O) components 718. A representative hardware layer 652 is illustrated and can represent, for example, the machine 700 of FIG. 7. The representative hardware layer 652 includes a processing unit 654 having associated executable instructions 604. Executable instructions 604 represent the executable instructions of the software architecture 606, including implementation of the methods, components, and so forth described herein. The hardware layer 652 also includes memory and/or storage devices memory/storage 656, which also have executable instructions 604. The hardware layer 652 may also comprise other hardware 658. The software architecture 606 may be deployed in any one or more of the components shown in FIG. 1. The software architecture 606 can be utilized to apply a machine learning technique or model to generate a prediction of a likelihood that a second server of a second adjudication entity outranks a set of fulfillment information of a first server of a first adjudication entity.

In the example architecture of FIG. 6, the software architecture 606 may be conceptualized as a stack of layers where each layer provides particular functionality. For example, the software architecture 606 may include layers such as an operating system 602, libraries 620, frameworks/middleware 618, applications 616, and a presentation layer 614. Operationally, the applications 616 and/or other components within the layers may invoke API calls 608 through the software stack and receive messages 612 in response to the API calls 608. The layers illustrated are representative in nature and not all software architectures have all layers. For example, some mobile or special purpose operating systems may not provide a frameworks/middleware 618, while others may provide such a layer. Other software architectures may include additional or different layers.

The operating system 602 may manage hardware resources and provide common services. The operating system 602 may include, for example, a kernel 622, services 624, and drivers 626. The kernel 622 may act as an abstraction layer between the hardware and the other software layers. For example, the kernel 622 may be responsible for memory management, processor management (e.g., scheduling), component management, networking, security settings, and so on. The services 624 may provide other common services for the other software layers. The drivers 626 are responsible for controlling or interfacing with the underlying hardware. For instance, the drivers 626 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 620 provide a common infrastructure that is used by the applications 616 and/or other components and/or layers. The libraries 620 provide functionality that allows other software components to perform tasks in an easier fashion than to interface directly with the underlying operating system 602 functionality (e.g., kernel 622, services 624 and/or drivers 626). The libraries 620 may include system libraries 644 (e.g., C standard library) that may provide functions such as memory allocation functions, string manipulation functions, mathematical functions, and the like. In addition, the libraries 620 may include API libraries 646 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 620 may also include a wide variety of other libraries 648 to provide many other APIs to the applications 616 and other software components/devices.

The frameworks/middleware 618 (also sometimes referred to as middleware) provide a higher-level common infrastructure that may be used by the applications 616 and/or other software components/devices. For example, the frameworks/middleware 618 may provide various graphic user interface functions, high-level resource management, high-level location services, and so forth. The frameworks/middleware 618 may provide a broad spectrum of other APIs that may be utilized by the applications 616 and/or other software components/devices, some of which may be specific to a particular operating system 602 or platform.

The applications 616 include built-in applications 638 and/or third-party applications 640. Examples of representative built-in applications 638 may include, but are not limited to, a contacts application, a browser application, a book reader application, a location application, a media application, a messaging application, and/or a game application. Third-party applications 640 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 640 may invoke the API calls 608 provided by the mobile operating system (such as operating system 602) to facilitate functionality described herein.

The applications 616 may use built-in operating system functions (e.g., kernel 622, services 624, and/or drivers 626), libraries 620, and frameworks/middleware 618 to create UIs to interact with users of the system. Alternatively, or additionally, in some systems, interactions with a user may occur through a presentation layer, such as presentation layer 614. In these systems, the application/component “logic” can be separated from the aspects of the application/component that interact with a user.

FIG. 7 is a block diagram illustrating components of a machine 700, according to some example embodiments, able to read instructions from a machine-readable medium (e.g., a machine-readable storage medium) and perform any one or more of the methodologies discussed herein. Specifically, FIG. 7 shows a diagrammatic representation of the machine 700 in the example form of a computer system, within which instructions 710 (e.g., software, a program, an application, an applet, an app, or other executable code) for causing the machine 700 to perform any one or more of the methodologies discussed herein may be executed. For example, the instructions 710 may be executed by the system 100 to transmit a communication to a second server associated with a second claim adjudication entity requesting alternate fulfillment information and select a claim adjudication process based on a comparison between initial fulfillment information and the alternate fulfillment information.

As such, the instructions 710 may be used to implement devices or components described herein. The instructions 710 transform the general, non-programmed machine 700 into a particular machine 700 programmed to carry out the described and illustrated functions in the manner described. In alternative embodiments, the machine 700 operates as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 710, sequentially or otherwise, that specify actions to be taken by machine 700. Further, while only a single machine 700 is illustrated, the term “machine” shall also be taken to include a collection of machines that individually or jointly execute the instructions 710 to perform any one or more of the methodologies discussed herein.

The machine 700 may include processors 704, memory/storage 706, and I/O components 718, which may be configured to communicate with each other such as via a bus 702. In an example embodiment, the processors 704 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 708 and a processor 712 that may execute the instructions 710. The term “processor” is intended to include multi-core processors 704 that may comprise two or more independent processors (sometimes referred to as “cores”) that may execute instructions contemporaneously. Although FIG. 7 shows multiple processors 704, the machine 700 may include a single processor with a single core, a single processor with multiple cores (e.g., a multi-core processor), multiple processors with a single core, multiple processors with multiple cores, or any combination thereof.

The memory/storage 706 may include a memory 714, such as a main memory, or other memory storage, database 152, and a storage unit 716, both accessible to the processors 704 such as via the bus 702. The storage unit 716 and memory 714 store the instructions 710 embodying any one or more of the methodologies or functions described herein. The instructions 710 may also reside, completely or partially, within the memory 714, within the storage unit 716, within at least one of the processors 704 (e.g., within the processor's cache memory), or any suitable combination thereof, during execution thereof by the machine 700. Accordingly, the memory 714, the storage unit 716, and the memory of processors 704 are examples of machine-readable media.

The I/O components 718 may include a wide variety of components to receive input, provide output, produce output, transmit information, exchange information, capture measurements, and so on. The specific I/O components 718 that are included in a particular machine 700 will depend on the type of machine. For example, portable machines such as mobile phones will likely include a touch input device or other such input mechanisms, while a headless server machine will likely not include such a touch input device. It will be appreciated that the I/O components 718 may include many other components that are not shown in FIG. 7. The I/O components 718 are grouped according to functionality merely for simplifying the following discussion and the grouping is in no way limiting. In various example embodiments, the I/O components 718 may include output components 726 and input components 728. The output components 726 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 728 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 718 may include biometric components 739, motion components 734, environmental components 736, or position components 738 among a wide array of other components. For example, the biometric components 739 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 734 may include acceleration sensor components (e.g., accelerometer), gravitation sensor components, rotation sensor components (e.g., gyroscope), and so forth. The environmental components 736 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 738 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 718 may include communication components 740 operable to couple the machine 700 to a network 737 or devices 729 via coupling 724 and coupling 722, respectively. For example, the communication components 740 may include a network interface component or other suitable device to interface with the network 737. In further examples, communication components 740 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 729 may be another machine or any of a wide variety of peripheral devices (e.g., a peripheral device coupled via a USB).

Moreover, the communication components 740 may detect identifiers or include components operable to detect identifiers. For example, the communication components 740 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 740, such as location via Internet Protocol (IP) geo-location, location via Wi-Fi® signal triangulation, location via detecting a NFC beacon signal that may indicate a particular location, and so forth.

FIG. 8 is a functional block diagram of an example neural network 802 that can be used for the inference engine or other functions (e.g., engines) as described herein to produce a predictive model. The predictive model can identify a likelihood that the second server outranks a set of fulfillment information of the first server. In an example, the neural network 802 can be a LSTM neural network. In an example, the neural network 802 can be a recurrent neural network (RNN). The example neural network 802 may be used to implement the machine learning as described herein, and various implementations may use other types of machine learning networks. The neural network 802 includes an input layer 804, a hidden layer 808, and an output layer 812. The input layer 804 includes inputs 804a, 804b . . . 804n. The hidden layer 808 includes neurons 808a, 808b . . . 808n. The output layer 812 includes outputs 812a, 812b . . . 812n.

Each neuron of the hidden layer 808 receives an input from the input layer 804 and outputs a value to the corresponding output in the output layer 812. For example, the neuron 808a receives an input from the input 804a and outputs a value to the output 812a. Each neuron, other than the neuron 808a, also receives an output of a previous neuron as an input. For example, the neuron 808b receives inputs from the input 804b and the output 812a. In this way the output of each neuron is fed forward to the next neuron in the hidden layer 808. The last output 812n in the output layer 812 outputs a probability associated with the inputs 804a-904n. Although the input layer 804, the hidden layer 808, and the output layer 812 are depicted as each including three elements, each layer may contain any number of elements. Neurons can include one or more adjustable parameters, weights, rules, criteria, or the like. These neurons can be adjusted based on the input and their adjustable nature.

In various implementations, each layer of the neural network 802 must include the same number of elements as each of the other layers of the neural network 802. For example, training patient information data features may be processed to create the inputs 804a-904n. The neural network 802 may implement a model to produce a prediction that a second server of a second adjudication entity includes fulfillment information that outranks fulfillment information of a first server of a first adjudication entity. More specifically, the inputs 804a-904n can include prescription drug features (binary, vectors, factors or the like).

The prescription drug features can be provided to neurons 808a-908n for analysis and connections between the known facts. The neurons 808a-908n, upon finding connections, provides the potential connections as outputs to the output layer 812, which determines a likelihood that a second server of a second adjudication entity includes fulfillment information for the prescription drug that outranks fulfillment information of a first server of a first adjudication entity.

The neural network 802 can perform any of the above calculations. The output of the neural network 802 can be used to trigger communication between the adjudication platform 150 and the second claim adjudication entity servers 160.

In some embodiments, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 804a is connected to each of neurons 808a, 808b . . . 808n.

Claims, in example embodiments, are data structures with individual fields therein that are adjudicated using one or more schemas to produce an output that may be shared with multiple parties, e.g., a pharmacy computer, a patient device, a data storage, a cloud storage or the like. In some instances a claim relates to healthcare or a regulated substance, e.g., a prescription drug. It is important that the schema adjudicate a claim in a manner of seconds to provide a timely reply to the waiting party devices. If the claim is to be processed by adding fourth party schemas, which may relate to cash price of prescriptions out of the normal schemas versus insured drug prices (inside normal schemas), significant delay in data processing can be incurred as a penalty. It is desirable to predict the result of a fourth party schema on the claim being processed and bypass such data processing if the delay penalty is too high or there is low likelihood that the fourth party schema will result in a better outcome for the patient. Various embodiments described herein provide these benefits to the data processing.

The claim data record, in an example embodiment, can include the information within the following categories: (a) definition data; (b) provider data; (c) facility data; (d) insurer data; (e) claim processing data; (f) claim facts data; (g) claimant data; (h) date data; and (i) claim resolution data. Additional data fields may include, a unique claim identifier, a claim status code, a provider identifier, an insurance identifier, a claimant identifier, a creation date, an update date, a financial amount claimed, and a financial amount approved. Each of these information elements can be fed to dedicated input nodes in a neural network.

Claim data records can also include data related to the claimant master record (e.g., a master record containing data related to a particular claimant) including, for example, the full name of the claimant, the address of the claimant, the date of birth of the claimant, unique identifiers for the claimant, the sex and/or gender of the claimant, other claimant details. In some cases, the claimant is not a named policy holder of the primary insurance and is, instead, a beneficiary of a covered policy holder. In such cases, the claimant data may also specify the full name of the policy holder, the address of the policy holder, the date of birth of the policy holder, unique identifiers for the policy holder, the sex and/or gender of the policy holder, the relationship between the policy holder and the claimant, and other policy holder details. However, in many cases such information regarding the claimant or the policy holder is incomplete or inaccurate.

Date data can also be included in the claim record and include time and date records associated with the claim identifying, for example, the date of the incident leading to the claim, the date of the treatment, the date of the filing of the claim, the date of each adjudication (if any), the date of the resolution of the claim (if any), and the date of the payment of the claim (if any).

A further example embodiment can include a computing system having a processor; and a memory operably in communication with the processor The memory has a set of instructions, which when executed by the processor, cause the computing system to identify a plurality of claim records that are associated with first heterogeneous data schemas, identify a plurality of coverage records that are associated with second heterogeneous data schemas, wherein the second heterogeneous data schemas are at least partially distinct from the first heterogeneous data schemas. In an example, the first data schema relates to an insurance claim record adjudication and the second data schema relates to a third party claim record adjudication. In an example, at least part of the first heterogeneous data schemas and the second heterogeneous data schemas are associated with a human-readable format. The instructions can further transform, with a joint schema, the plurality of claim records from the first heterogeneous data schemas and the plurality of coverage records from the second heterogeneous data schemas to a homogeneous data schema to generate transformed claim records and transformed coverage records. In an example, the homogeneous data schema is associated with a machine-readable format. The instructions can further identify a qualifying claim from a first transformed claim record of the transformed claim records and predict that the qualifying claim results in an possible approved adjudication by the fourth party. The instructions further integrate and report one of the two adjudication results.

### Glossary

“CARRIER SIGNAL” in this context refers to any intangible medium that is capable of storing, encoding, or carrying transitory or non-transitory instructions for execution by the machine, and includes digital or analog communications signals or other intangible medium to facilitate communication of such instructions. Instructions may be transmitted or received over the network using a transitory or non-transitory transmission medium via a network interface device and using any one of a number of well-known transfer protocols. The instructions can include claim records that are started at a provider device or at a pharmacy device. The claim records can be electrically transmitted on a carrier signal.

“CLIENT DEVICE” in this context refers to any machine that interfaces to a communications network to obtain resources from one or more server systems or other client devices. A client device may be, but is not limited to, a mobile phone, desktop computer, laptop, PDA, smart phone, tablet, ultra-book, netbook, laptop, multi-processor system, microprocessor-based or programmable consumer electronics, game console, set-top box, or any other communication device that a user may use to access a network.

“COMMUNICATIONS NETWORK” in this context refers to one or more portions of a network that may be an ad hoc network, an intranet, an extranet, a virtual private network (VPN), a LAN, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an ASIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC, or any combination thereof. A processor may further be a multi-core processor having two or more independent processors (sometimes referred to as “cores”) that may execute instructions contemporaneously.

“TIMESTAMP” in this context refers to a sequence of characters or encoded information identifying when a certain event occurred, for example giving date and time of day, sometimes accurate to a small fraction of a second.

Changes and modifications may be made to the disclosed embodiments without departing from the scope of the present disclosure. These and other changes or modifications are intended to be included within the scope of the present disclosure, as expressed in the following claims.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

What is claimed is:

1. A method comprising:
receiving, by a first server associated with a first claim adjudication entity, a claim for a prescription drug for a patient, the first claim adjudication entity managing a prescription drug profile for the patient;
accessing initial fulfillment information for fulfilling the prescription drug using the first claim adjudication entity;
determining that one or more features associated with the prescription drug of the claim correspond to one or more criteria;
in response to determining that one or more features associated with the prescription drug of the claim correspond to the one or more criteria, transmitting a communication to a second server associated with a second claim adjudication entity, the communication identifying the prescription drug and requesting alternate fulfillment information; and
selecting a claim adjudication process based on the initial fulfillment information and the alternate fulfillment information.

2. The method of claim 1, wherein the determining that the one or more features correspond to the one or more criteria comprises:
receiving input from the patient for opting into an alternate claim adjudication process comprising communication with the second server of the second claim adjudication entity.

3. The method of claim 2, further comprising:
causing presentation, on a client device of the patient, of a list of alternate claim adjudication entities comprising at least the second claim adjudication entity; and
receiving, from the client device, a selection of a subset of the alternate claim adjudication entities that are on the list.

4. The method of claim 3, further comprising:
in response to determining that one or more features associated with the prescription drug of the claim correspond to the one or more criteria, transmitting a plurality of communications to each server of the subset of the alternate claim adjudication entities indicated in the selection received from the client device, each of the communications identifying the prescription drug and requesting alternate fulfillment information.

5. The method of claim 1, wherein the determining that the one or more features correspond to the one or more criteria comprises:
applying a model to the one or more features associated with the prescription drug to rank the prescription drug; and
determining that the rank transgresses a threshold rank.

6. The method of claim 5, wherein the model comprises a machine learning model comprising a neural network, the machine learning model trained to establish a relationship between a plurality of prescription drug features and likelihoods indicating that a set of alternate fulfillment information of the second server outranks a set of fulfillment information of the first server.

7. The method of claim 6, further comprising training the machine learning model by performing operations comprising:
obtaining a batch of training data comprising a first set of the plurality of training prescription drug features and ground truth likelihoods indicating that the set of alternate fulfillment information of the second server outranks the set of fulfillment information of the first server;
processing the first set of the plurality of training prescription drug features by the machine learning model to generate an estimated rank;
computing a loss based on a deviation between the estimated rank and the ground truth likelihoods; and
updating one or more parameters of the machine learning model based on the computed loss.

8. The method of claim 1, wherein the determining that the one or more features correspond to the one or more criteria comprises:
accessing a predetermined list of prescription drugs available for fulfillment by the second claim adjudication entity; and
determining that an identifier represented by the one or more features of the prescription drug matches one or more identifiers on the predetermined list of prescription drugs.

9. The method of claim 1, further comprising:
initializing a timer in response to transmitting the communication to the second server.

10. The method of claim 9, further comprising:
determining that a response from the second server comprising the alternate fulfillment information has been received prior to expiration of the timer, wherein the selecting the claim adjudication process based on the initial fulfillment information and the alternate fulfillment information is performed in response to determining that the response has been received prior to the expiration of the timer.

11. The method of claim 9, further comprising:
determining that a response from the second server comprising the alternate fulfillment information has failed to be received prior to expiration of the timer, wherein the selecting the claim adjudication process based on the initial fulfillment information and the alternate fulfillment information selects the initial fulfillment information in response to determining that the response has failed to be received prior to the expiration of the timer.

12. The method of claim 1, further comprising:
identifying one or more peak times for communicating with the second server;
determining that a current time falls within the one or more peak times; and
conditioning transmission of the communication to the second server in response to determining that the current time falls within the one or more peak times.

13. The method of claim 1, further comprising:
comparing the initial fulfillment information with the alternate fulfillment information;
determining that the alternate fulfillment information is associated with a higher priority than the initial fulfillment information in response to comparing the initial fulfillment information with the alternate fulfillment information; and
in response to determining that the alternate fulfillment information is associated with the higher priority, selecting, as the claim adjudication process, operations for adjudicating the claim for the prescription drug via the second claim adjudication entity.

14. The method of claim 13, further comprising:
utilizing the prescription drug profile for the patient by the first claim adjudication entity in response to adjudicating the claim for the prescription drug via the second claim adjudication entity.

15. The method of claim 14, further comprising:
storing an identification of the prescription drug in the prescription drug profile; and
determining drug interaction information between the prescription drug and other drugs listed in the prescription drug profile.

16. The method of claim 1, further comprising transmitting the communication from the first server to the second server via an intermediate hub.

17. The method of claim 1, wherein the prescription drug profile comprises at least one of an electronic health record, past claims information for the patient, patient health information, past medical recommendations, past treatment recommendations, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, or one or more treatment preferences.

18. The method of claim 1, further comprising:
receiving the alternate fulfillment information from the second server; and
modifying the initial fulfillment information based on the alternate fulfillment information.

19. A system comprising:
one or more processors coupled to a memory comprising non-transitory computer instructions that when executed by the one or more processors perform operations comprising:
receiving, by a first server associated with a first claim adjudication entity, a claim for a prescription drug for a patient, the first claim adjudication entity managing a prescription drug profile for the patient;
accessing initial fulfillment information for fulfilling the prescription drug using the first claim adjudication entity;
determining that one or more features associated with the prescription drug of the claim correspond to one or more criteria;
in response to determining that one or more features associated with the prescription drug of the claim correspond to the one or more criteria, transmitting a communication to a second server associated with a second claim adjudication entity, the communication identifying the prescription drug and requesting alternate fulfillment information; and
selecting a claim adjudication process based on the initial fulfillment information and the alternate fulfillment information.

20. A non-transitory computer readable medium comprising non-transitory computer-readable instructions for performing operations comprising:
receiving, by a first server associated with a first claim adjudication entity, a claim for a prescription drug for a patient, the first claim adjudication entity managing a prescription drug profile for the patient;
accessing initial fulfillment information for fulfilling the prescription drug using the first claim adjudication entity;
determining that one or more features associated with the prescription drug of the claim correspond to one or more criteria;
in response to determining that one or more features associated with the prescription drug of the claim correspond to the one or more criteria, transmitting a communication to a second server associated with a second claim adjudication entity, the communication identifying the prescription drug and requesting alternate fulfillment information; and
selecting a claim adjudication process based on the initial fulfillment information and the alternate fulfillment information.
